(19) United States
(12) Patent Application Publication
(10) Pub. No.: US 2016/0199315 A1
(43) Pub. Date: Jul. 14, 2016

(54) ADHESIVE PATCH

(71) Applicant: HISAMITSU PHARMACEUTICAL CO., INC., Tosu-shi, Saga (JP)

(72) Inventors: Yuka TAKAGI, Tsukuba-shi, Ibaraki (JP); Yasunori TAKADA, Tsukuba-shi, Ibaraki (JP)

(73) Assignee: HISAMITSU PHARMACEUTICAL CO., INC., Tosu-shi, Saga (JP)

(21) Appl. No.: 14/913,472

(22) PCT Filed: Aug. 6, 2014
(86) PCT No.: PCT/JP2014/070770

§ 371 Date: Feb. 22, 2016

(30) Foreign Application Priority Data
Aug. 23, 2013 (JP) 2013-173172

(51) Int. Cl. A61K 9/70; A61K 47/32; A61K 47/20; A61K 47/06; A61K 47/14; A61K 47/10; A61K 31/439; A61K 47/12

(52) U.S. Cl. CPC A61K 9/7061; A61K 31/439; A61K 47/32; A61K 47/12; A61K 9/7053; A61K 47/06; A61K 47/14; A61K 47/10; A61K 47/20

(57) ABSTRACT

The present invention is a patch comprising a support, and an adhesive layer disposed on at least one surface of the support and formed of an adhesive composition comprising a drug and an adhesive, wherein the drug is cytisine or a salt thereof, and the adhesive composition has an acid value of 10 or less.

### TECHNICAL FIELD

The present invention relates to patches.

### BACKGROUND ART

Cytisine is a partial agonist binding to nicotinic acetylcholine receptor subtype (14132 with high affinity. Cytisine has been used in the former socialist economy countries for the purpose of smoking cessation. Cytisine is administrated 6 times per day in the form of a tablet (for example, Non Patent Literature 1).

### CITATION LIST

### Non Patent Literature

Non Patent Literature 1: The New England Journal of Medicine, 2011, Vol. 365, p. 1193-1200

### SUMMARY OF INVENTION

### Technical Problem

The cytisine tablet requires oral administration 6 times per day, which is not preferred in view of compliance of patients.

Then, an object of the present invention is to provide a patch having good releasing property of cytisine from an adhesive layer, and enabling reduction in the number of administrations of cytisine per day.

### Solution to Problem

The present invention provides a patch comprising a support, and an adhesive layer disposed on at least one surface of the support and formed of an adhesive composition comprising a drug and an adhesive, wherein the drug is cytisine or a salt thereof, and the adhesive composition has an acid value of 10 or less.

The patch according to the present invention has not only good property of releasing cytisine from the adhesive layer (releasing property), but also high stability of cytisine in the adhesive layer and high skin permeability of cytisine. Accordingly, the number of administrations of cytisine per day can be reduced, which improves compliance of patients.

In the patch according to the present invention, the adhesive may be an acrylic adhesive. If the adhesive is the acrylic adhesive, the releasing property of cytisine from the adhesive layer and the skin permeability of cytisine are enhanced. Among these acrylic adhesives, if a hydroxyl group-containing acrylic adhesive is used, enhancement in releasing property of cytisine from the adhesive layer is remarkable.

In the patch according to the present invention, the adhesive may be a rubber adhesive. If the adhesive is the rubber adhesive, the stability of cytisine in the adhesive layer is enhanced. The term “rubber adhesive” indicates “adhesive containing a homo- or copolymer of monomer having a conjugated double bond.” If an organic acid or a salt thereof is comprised in the adhesive composition, the releasing property of cytisine from the adhesive layer is more significantly enhanced.

In the patch according to the present invention, the adhesive composition may further comprise an absorption enhancer. If the absorption enhancer is comprised in the adhesive composition, the skin permeability of cytisine is enhanced.

### Advantageous Effects of Invention

According to the present invention, a patch having good releasing property of cytisine from the adhesive layer is provided.

### DESCRIPTION OF EMBODIMENTS

Next, an embodiment of the patch according to the present invention will be described in detail.

The patch according to an embodiment of the present invention includes a support. As the support, a stretchable or non-stretchable sheet, film, or foil can be used. The material for the support is not limited in particular; examples thereof include polymer such as polyester (poly(ethylene terephthalate), poly(butylene terephthalate), poly(ethylene naphthalate), etc.), polyolefin (polyethylene, polypropylene, etc.), polybutadiene, ethylene-vinyl acetate copolymer, poly(vinyl chloride), nylon, or polyurethane; paper; or metal such as aluminum. This may be provided in the form of woven fabric or non-woven fabric. The support may be a laminate, a foamed body, or a microporous body.

The patch includes an adhesive layer disposed on at least one surface of the support. The adhesive layer may be disposed only on one surface of the support, or may be disposed on both surfaces of the support. Furthermore, the adhesive layer may be disposed across one surface or both surfaces of the support, or may be disposed on part of the surface(s) of the support.

The adhesive layer is formed of an adhesive composition, and the acid value of the adhesive composition is 10 or less. Because the acid value of the adhesive composition is 10 or less, the patch has not only good releasing property of cytisine from the adhesive layer but also high stability of cytisine in the adhesive layer and high skin permeability of cytisine. An acid value of 5 or less is preferred, and an acid value of 3 or less is more preferred.

Here, the acid value is defined as follows.

An amount of 0.4 g of the adhesive composition is weighed, and is placed in a 50 mL centrifuge tube; 20 mL of a mixed solution of toluene and ethanol in a volume ratio of 1:1 is added to dissolve the adhesive composition. Next, 0.5 mL of a phenolphthalein indicator is added thereto, titration is performed using a solution of 0.05 mol/L, potassium hydroxide in ethanol.

On the other hand, a blank test is performed in the same manner as above except that the adhesive composition is not used. The amount of potassium hydroxide in milligram needed for neutralization of 1 g of the adhesive composition is calculated from the titer of the solution of 0.05 mol/L potassium hydroxide in ethanol, which is a correction of the result of the blank test, and is defined as an acid value.

The acid value of the adhesive composition is controlled to be 10 or less by adding an acid neutralizer to the adhesive composition when necessary. The acid neutralizer is not limited in particular; examples thereof include alkali metal hydroxide or alkaline earth metal hydroxide such as sodium hydroxide, potassium hydroxide, magnesium hydroxide, or calcium hydroxide; ammonia; or amine.

The adhesive composition comprises cytisine or a salt thereof as a drug. Cytisine or a salt thereof is used as smoking cessation aid in smoking cessation of smokers and treatment of patients with nicotine addiction.

The salt of cytisine is usually pharmaceutically permissible salt. Examples of the salt include inorganic salt such as hydrochloride, hydrobromate, hydroiodide, sulfate, nitrate, or phosphate; or organic acid salt such as acetate, citrate, maleate, malate, succinate, oxalate, tartrate, or lactate.

Although the content of cytisine or a salt thereof is set according to the purpose such as smoking cessation for smokers or treatment of patients with nicotine addiction, the content is preferably 0.5 to 20% by weight, more preferably 1 to 10% by weight, particularly preferably 2 to 6% by weight relative to the total weight of the adhesive composition. If the content of cytisine or a salt thereof is 2 to 6% by weight relative to the total weight of the adhesive composition, the area of the patch is about 20 to 40 cm² based on the cumulative permeation amount (C.A. (μg/cm²)) of cytisine in the patch (Example 11). If the bioavailability of the patch is 30 to 100%, it is desirable that the content of cytisine or a salt thereof be 0.5 to 5% by weight relative to the total weight of the adhesive composition.

The adhesive composition comprises an adhesive. The “adhesive” has the same meaning as that of “pressure-sensitive adhesive.”

The adhesive is not limited in particular; examples thereof include acrylic adhesive, rubber adhesive, silicone adhesive, urethane adhesive, vinyl ether adhesive, or isobutylene adhesive. As the adhesive, acrylic adhesive or rubber adhesive is preferred; if the adhesive is acrylic adhesive, the releasing property of cytisine from the adhesive layer and the skin permeability of cytisine are enhanced; if the adhesive is rubber adhesive, the stability of cytisine in the adhesive layer is enhanced.

The “acrylic adhesive” indicates “adhesive comprising a polymerization product of monomers comprising a (meth)acryloyl skeleton-containing monomer as a monomer component”; it is preferred that the (meth)acryloyl skeleton-containing monomers comprise a (meth)acrylate ester. It is preferred that the acrylic adhesive be an adhesive comprising a homo- or copolymer of alkyl (meth)acrylate ester. The term “(meth)acrylic” indicates “acrylic” or “methacrylic.” The copolymer of alkyl (meth)acrylate ester may be a copolymer of two or more different alkyl (meth)acrylate esters, or may be a copolymer of one or two or more different alkyl (meth)acrylate ester(s) and the other monomer(s). As the alkyl (meth)acrylate ester, C4-16 alkyl (meth)acrylate ester is preferred, and butyl (meth)acrylate, isobutyl (meth)acrylate, hexyl (meth)acrylate, 2-ethylhexyl (meth)acrylate, octyl (meth)acrylate, isooctyl (meth)acrylate, isononyl (meth)acrylate, or decyl (meth)acrylate is more preferred. Examples of the other monomer include hydroxyalkyl (meth)acrylate ester (such as hydroxyethyl (meth)acrylate or hydroxypropyl (meth)acrylate), styrene, methylstyrene, N-vinylpyrrolidone, (meth)acrylamide, or vinyl acetate.

Among these acrylic adhesives, hydroxyl group-containing acrylic adhesive is preferred. As the “hydroxyl group-containing acrylic adhesive,” the adhesive comprising a copolymer of alkyl (meth)acrylate ester and hydroxyalkyl (meth)acrylate ester is preferred. The “hydroxyl group-containing acrylic adhesive” may be an adhesive comprising a copolymer composed of alkyl (meth)acrylate ester, hydroxyalkyl (meth)acrylate ester, and the other monomer. Here, the other monomer may be two or more different monomers. As the other monomer, vinyl acetate is preferred. In particular, an adhesive comprising a copolymer composed of monomers comprising 2-ethylhexyl acrylate, hydroxyethyl acrylate, and vinyl acetate can be used as the “hydroxyl group-containing acrylic adhesive.” If a hydroxyl group-containing acrylic adhesive is used, enhancement in the releasing property of cytisine from the adhesive layer is remarkable.

As the hydroxyl group-containing acrylic adhesive, Duro-TAK 87-2510, Duro-TAK 87-202A, Duro-TAK 87-4287, Duro-TAK 87-2287, Duro-TAK 87-2516, or Duro-TAK 87-2525 (manufactured by Henkel AG & Co. KGaA) is available; as another acrylic adhesive, Duro-TAK 87-900A, Duro-TAK 87-4098, Duro-TAK 87-2100, or Duro-TAK 87-9301 (all manufactured by Henkel AG & Co. KGaA) is available.

The rubber adhesive indicates “adhesive comprising a homo- or copolymer of monomer having a conjugated double bond.” The homo- or copolymer of monomer having a conjugated double bond also includes natural rubber or reproduced rubber.

Examples of the homo- or copolymer of monomer having a conjugated double bond include styrene-butadiene copolymer, acrylonitrile-butadiene copolymer, butadiene polymer, isoprene polymer, chloroprene polymer, isobutylene-isoprene copolymer, and styrene-isoprene copolymer.

Among these homo- or copolymers, styrene-butadiene copolymer such as styrene-butadiene-styrene block copolymer, or styrene-isoprene copolymer such as styrene-isoprene-styrene block copolymer is preferred, and styrene-isoprene-styrene block copolymer (SISs) is most preferred.

As the styrene-isoprene-styrene block copolymer, SIS5002 (manufactured by JSR Corporation), Quintac 3530, Quintac 3421, and Quintac 3570C (manufactured by ZEON Corporation), and Kraton D-KX401CS and Kraton D-1107CU (manufactured by Kraton Performance Polymers, Inc.) can be preferably used.

The adhesive may comprise a tackifier, a plasticizer, a filler, an anti-aging agent (stabilizer), or a crosslinking agent as a component.

As the tackifier, alicyclic hydrocarbon resin, rosin resin, terpene resin, petroleum resin, phenol resin, or xylene resin can be used. In particular, alicyclic hydrocarbon resin is preferred. The rubber adhesive usually comprises a tackifier as a component. The tackifier may be used singly or in combinations of two or more.

Examples of the plasticizer include petroleum oil such as paraffin process oil, naphthene process oil, or aromatic process oil; vegetable oil such as olive oil, camellia oil, castor oil, tall oil, or peanut oil; dibasic acid ester such as dibutyl phthalate or dioctyl phthalate; liquid rubber such as liquid polybutene or liquid isoprene; polyhydric alcohol such as diethylene glycol, polyethylene glycol, propylene glycol, or dipropylene glycol; squalane; or squalene. In particular, use of a liquid paraffin as a paraffin process oil, or a liquid polybutene as a liquid rubber is preferred. This may be used singly or in combinations of two or more.

Examples of the filler include aluminum hydroxide, calcium carbonate, magnesium carbonate, silicic acid or silicate, barium sulfate, calcium sulfate, calcium plumbite, zinc oxide, and titanium oxide.

Examples of the anti-aging agent (stabilizer) include antioxidant, or ultraviolet absorbing agent such as p-aminobenzoic acid derivative, anthranilic acid derivative, salicylic acid derivative, coumarin derivative, imidazoline derivative, pyrimidine derivative, or dioxane derivative.

The adhesive may be used singly or in combinations of two or more. The amount of the adhesive to be compounded is preferably 50 to 99.5% by weight, more preferably 70 to 99% by weight, particularly preferably 80 to 98% by weight relative to the total weight of the adhesive composition.

The adhesive composition may comprise, in addition to cytisine or a salt thereof and an adhesive, further an organic acid or a salt thereof. In particular, if an organic acid or a salt thereof is comprised in the adhesive composition in which the adhesive is a rubber adhesive, the releasing property of cytisine from the adhesive layer is more significantly enhanced.

The organic acid usually has a carboxyl group; examples of the organic acid include acidic amino acid such as aspartic acid or glutamic acid; saturated or unsaturated fatty acid such as valeric acid, caprylic acid, capric acid, lauric acid, myristic acid, palmitic acid, stearic acid, isostearic acid, or oleic acid; aromatic carboxylic acid such as benzoic acid; aliphatic hydroxy acid such as lactic acid, tartaric acid, malic acid, and citric acid; dicarboxylic acid such as malonic acid, succinic acid, glutaric acid, adipic acid, fumaric acid, or maleic acid; (meth)acrylic polymer such as poly(meth)acrylic acid; polysaccharide having carboxyl groups such alginic acid; or ascorbic acid.

The salt of the organic acid is not particularly limited; examples thereof include alkali metal salt such as sodium salt or potassium salt; or alkaline earth metal salt such as magnesium salt or calcium salt.

The organic acid or the salt thereof may be used singly or in combinations of two or more. The amount of the organic acid or the salt thereof to be compounded is preferably 0.5 to 10% by weight, preferably 1 to 7% by weight relative to the total weight of the adhesive composition.

If an organic acid or a salt thereof, particularly an organic acid is comprised in the adhesive composition, the acid value of the adhesive composition should not exceed 10. If the acid value exceeds 10 by the organic acid comprised in the adhesive composition, the acid neutralizer described above is comprised in the adhesive composition.

The adhesive composition may further comprise an absorption enhancer. The absorption enhancer can enhance the absorption of cytisine or a salt thereof. If the absorption enhancer is comprised in the adhesive composition, the skin permeability of cytisine in the patch is enhanced.

Examples of the absorption enhancer can include higher aliphatic alcohol such as lauryl alcohol, oleyl alcohol, octyldodecanol, stearyl alcohol, isostearyl alcohol, myristyl alcohol, or cetanol; higher fatty acid such as myristic acid, lauric acid, palmitic acid, stearic acid, oleic acid, or linoleic acid; higher fatty acid ester such as methyl laurate, hexyl laurate, isopropyl palmitate, isopropyl myristate, myristyl myristate, octyldodecyl myristate, or cetyl palmitate; dicarboxylic acid diester such as diethyl sebacate or diisopropyl sebacate; tricarboxylic acid triester such as triethyl citrate; aromatic carboxylic acid ester such as methyl salicylate, glycol salicylate, or ethyl salicylate; higher fatty acid ester of polyhydric alcohol such as glycerol monocaprylate, glycerol monolaurate, glycerol monooleate, sorbitan monolaurate, sorbitan monooleate, sucrose monolaurate, propylene glycol monolaurate, polyethylene glycol monolaurate, or polyethylene glycol monostearate; terpene; triacetin; N-methyl-2-pyrrolidone; crotamiton; polyhydric alcohol such as propylene glycol, dipropylene glycol, or polyethylene glycol; 1-dodecylazacycloheptan-2-one (Azone); or dimethyl sulfoxide. In particular, higher aliphatic alcohol, higher fatty acid ester, dicarboxylic acid diester, or mono higher fatty acid ester of polyhydric alcohol is preferred; if it is comprised in the adhesive composition, the skin permeability of cytisine is significantly enhanced. If a higher fatty acid is comprised in the adhesive composition as the absorption enhancer, the acid value of the adhesive composition should not exceed 10.

The absorption enhancer may be used singly or in combinations of two or more. The amount of the absorption enhancer to be compounded is preferably 1 to 20% by weight, preferably 3 to 10% by weight relative to the total weight of the adhesive composition.

Although the thickness of the adhesive layer formed of the adhesive composition comprising the components described above can be any thickness allowing application to the skin and release of cytisine from the adhesive layer, the thickness is usually 10 to 1000 μm, preferably 30 to 500 μm, more preferably 50 to 200 μm.

A separating material may be disposed on the adhesive layer of the patch to protect the adhesive layer.

The separating material is usually a sheet, a film, or a foil. The material for the separating material may be any material which can be peeled off from the adhesive layer in use of the patch; examples of the material include polyester such as poly(ethylene terephthalate), polybutylene terephthalate), or poly(ethylene naphthalate); polyolefin such as polyethylene or polypropylene; paper; or metal such as aluminum. The separating material may be a laminate.

It is preferred that the surface of the separating material be subjected to release treatment with silicone or polytetrafluoroethylene. The separating material can be readily peeled off from the adhesive layer by the release treatment.

The patch can have any shape and any size. Examples of the shape for the patch include rectangular, square, circular, or oval shape.

The patch can be produced as follows.

First, an adhesive composition is prepared. Cytisine or a salt thereof, an adhesive, and the other component(s) when necessary are dissolved or dispersed in a solvent with a mixer to prepare a solution or a dispersion liquid of the adhesive composition.

As the solvent, aromatic hydrocarbon such as toluene or xylene; aliphatic hydrocarbon such as hexane or heptane; alicyclic hydrocarbon such as cyclohexane; acetate ester such as ethyl acetate or butyl acetate; and alkanol (aliphatic alcohol) such as methanol, ethanol, or isopropanol can be used. The solvent may be used singly or in combinations of two or more.

Subsequently, the solution or the dispersion liquid of the adhesive composition is applied onto a support, and the solvent is volatilized to form an adhesive layer; or the solution or the dispersion liquid of the adhesive composition is applied onto a paper or a film subjected to release treatment, the solvent is volatilized to form an adhesive layer, a support is placed on the adhesive layer, the adhesive layer is transferred by pressing, and the paper or the film subjected to separating treatment is peeled off to form an adhesive layer on the support. A separating material is then disposed on the adhesive layer to prepare a patch.

Finally, a method of using a patch will be described. The patch is used in smoking cessation of smokers and treatment of patients with nicotine addiction. In use, the adhesive layer of the patch is applied to the skin of the upper arm, the belly, the lower back, or the upper back (dorsal region). If the patch includes a separating material, it is needless to say that the separating material is peeled off from the adhesive layer, and the adhesive layer is applied to the skin.

The patch is applied to the skin several times a day, preferably once a day, and has high compliance.

### EXAMPLES

Hereinafter, the present invention will be described in detail based on Examples, but the present invention will not be limited to these.

The acid value, the releasing property, the stability, and the skin permeability of the patch were measured and calculated as follows.

(Acid Value)

An amount of 0.4 g of an adhesive composition was weighed, and was placed in a 50 mL centrifuge tube; 20 mL of a mixed solution of toluene and ethanol at a volume ratio of 1:1 was added, and the adhesive composition was dissolved. Next, 0.5 mL of a phenolphthalein indicator as an indicator was added thereto, and titration was performed using a solution of 0.05 mol/L potassium hydroxide in ethanol.

A blank test was performed in the same manner as above except that the adhesive composition was not used. The amount of potassium hydroxide in milligram needed for neutralization of 1 g of the adhesive composition was calculated from the titer of the solution of 0.05 mol/L potassium hydroxide in ethanol, which was a correction of the result of the blank test, and was defined as an acid value (mgKOH/g).

(Releasing Property)

A patch was mounted on a rotary cylinder of a dissolution tester such that the adhesive layer faced the outside. Subsequently, a round-bottomed flask with 900 ml of a phosphoric acid buffer saline having a pH of 7.4 was mounted on the dissolution tester, and the temperature was set at 32° C. Next, the rotary cylinder was immersed in purified water in a round-bottomed flask, and was rotated at a rate of 50 rpm. Subsequently, 10 ml of the dissolution liquid was sampled at intervals of a predetermined time; the amount of cytisine released, which was measured by high-performance liquid chromatography, was divided by the cytisine content in the patch to calculate the release rate. The release rate after 24 hours is “24 hr release rate into water (%).”

(Stability)

A patch stamped out into a size of 3 cm² was placed in a 50 mL-volume centrifuge tube, and 10 mL of tetrahydrofuran was added thereto to dissolve the adhesive composition. A mixed solution of water/methanol at a volume ratio of 1:1 was added thereto, and the total volume was adjusted to 50 ml; then, the cytisine content was measured by high-performance liquid chromatography.

The cytisine content was measured by the above method before and after the patch was preserved under conditions at 60° C. and a humidity of 75% for two weeks, and the proportion of cytisine content after preservation (to the initial content %) was calculated where the cytisine content before preservation was 100%.

(Skin Permeability)

The skin of the body of a hairless mouse was peeled, and fat was removed. A patch was applied to the outer surface of the skin, and the resultant skin was set to a transparent test cell of flow-through type so as to contact the dermis with a receptor solution. The transparent test cell was filled with a receptor solution (phosphoric acid buffer saline having a pH of 7.4); circulating water heated so as to keep the receptor solution at 32° C. was circulated in the outer periphery; the receptor solution was fed at a flow rate of about 2.5 mL/hr, and sampling was performed every four hours until 24 hours. The cytisine content in the sampled receptor solution was measured by high-performance liquid chromatography to calculate the skin permeability of cytisine per hour (C.A. (μg/cm²)).

### Example 1

After cytisine and ethyl acetate (solvent) were preliminarily mixed with a mixer, a solution of hydroxyl group-containing acrylic adhesive Duro-TAK 87-2510 (manufactured by Henkel AG & Co, KGaA) was added thereto and mixed to prepare an adhesive composition solution. This adhesive composition solution was spread over a film subjected to release treatment; the solvent was removed by drying to form an adhesive layer having a thickness of 100 μm; a support was placed on the adhesive layer; and the adhesive layer was transferred by pressing to obtain a patch. The adhesive layer of the patch comprises 3% by weight of cytisine and 97% by weight of the adhesive relative to the total weight of the adhesive composition.

### Example 2

A patch was obtained in the same manner as in Example 1 except that Duro-TAK 87-2510 was replaced by hydroxyl group-containing acrylic adhesive Duro-TAK 87-202A (manufactured by Henkel AG & Co. KGaA).

### Example 3

A patch was obtained in the same manner as in Example 1 except that Duro-TAK 87-2510 was replaced by hydroxyl group-containing acrylic adhesive Duro-TAK 87-4287 (manufactured by Henkel AG & Co. KGaA).

### Example 4

After cytisine and toluene (solvent) were preliminarily mixed with a mixer, a mixed solution of styrene-isoprene-styrene block copolymer SIS5002 (manufactured by JSR Corporation), an alicyclic hydrocarbon resin, a liquid paraffin, and toluene, which was separately prepared, was added thereto and mixed to prepare an adhesive composition solution. This adhesive composition solution was spread over a film subjected to release treatment, the solvent was removed by drying to form an adhesive layer having a thickness of 100 μm; a support was placed on the adhesive layer, and the adhesive layer was transferred by pressing to obtain a patch. The adhesive layer of the patch comprises 3% by weight of cytisine and 97% by weight of the adhesive (SIS5002: 28.5% by weight, alicyclic hydrocarbon resin: 51.4% by weight, liquid paraffin: 17.1% by weight) relative to the total weight of the adhesive composition.

### Example 5

An patch was obtained in the same manner as in Example 1 except that Duro-TAK 87-2510 was replaced by acrylic adhesive Duro-TAK 87-900A having no hydroxyl group and no carboxyl group (manufactured by Henkel AG & Co. KGaA).

### Comparative Example 1

A patch was obtained in the same manner as in Example 4 except that a methacrylic acid copolymer as an acid was added to the adhesive composition solution in Example 4. The adhesive layer of the patch comprises 3% by weight of cytisine, 94% by weight of the adhesive (SIS5002: 27.7% by weight, alicyclic hydrocarbon resin: 49.8% by weight, liquid paraffin: 16.5% by weight), and 3% by weight of the methacrylic acid copolymer relative to the total weight of the adhesive composition.

### Comparative Example 2

A patch was obtained in the same manner as in Comparative Example 1 except that the methacrylic acid copolymer was replaced by valeric acid. The adhesive layer of the patch comprises 3% by weight of valeric acid rather than the methacrylic acid copolymer.

### Comparative Example 3

A patch was obtained in the same manner as in Comparative Example 1 except that the methacrylic acid copolymer was replaced by benzoic acid. The adhesive layer of the patch comprises 3% by weight of benzoic acid rather than the methacrylic acid copolymer.

### Comparative Example 4

A patch was obtained in the same manner as in Example 1 except that Duro-TAK 87-2510 was replaced by carboxyl group-containing acrylic adhesive Duro-TAK 387-2051 (manufactured by Henkel AG & Co. KGaA).

### Comparative Example 5

A patch was obtained in the same manner as in Example 1 except that Duro-TAK 87-2510 was replaced by carboxyl group-containing acrylic adhesive Duro-TAK 87-2194 (manufactured by Henkel AG & Co. KGaA).

The acid value, the releasing property, the stability, and the skin permeability of the patches in Examples 1 to 5 and Comparative Examples 1 to 5 are shown in Table 1.

TABLE 1

| | | | Releasing | | |
|---|---|---|---|---|---|
| | | | property | Stability | |
| | | | (24 hr | (to | Skin |
| | | | release rate | initial | permeability |
| | | Acid value | into water | content | (C.A. |
| | Adhesive | (mgKOH/g) | (%)) | %) | (μg/cm²)) |
| Example 1 | Duro-TAK 87-2510 | 1 | 55.1 | 97.7 | 78.73 |
| | Functional group: Hydroxyl group | | | | |
| Example 2 | Duro-TAK 87-202A | 3 | 109.5 | 96.8 | — |
| | Functional group: Hydroxyl group | | | | |
| Example 3 | Duro-TAK 87-4287 | 2 | 44.8 | 93.8 | — |
| | Functional group: Hydroxyl group | | | | |
| Example 4 | SIS5002 | 0 | 14.3 | 99.8 | 37.50 |
| | Functional group: None | | | | |
| Example 5 | Duro-TAK 87-900A | 2 | 21.1 | 96.3 | 60.19 |
| | Functional group: None | | | | |
| Comparative | SIS5002 | 15 | 6.9 | 100.9 | — |
| Example 1 | Functional group: None | | | | |
| Comparative | SIS5002 | 25 | 7.0 | 27.7 | — |
| Example 2 | Functional group: None | | | | |
| Comparative | SIS5002 | 40 | 5.1 | 97.6 | — |
| Example 3 | Functional group: None | | | | |
| Comparative | Duro-TAK 387-2051 | 98 | 2.6 | 89.9 | — |
| Example 4 | Functional group: COOH group | | | | |
| Comparative | Duro-TAK 87-2194 | 97 | 1.1 | 99.2 | — |
| Example 5 | Functional group: COOH group | | | | |

Evidently from Table 1, the patches in Examples 1 to 5 having an acid value of 10 or less have good releasing property of cytisine from the adhesive layer, high stability of cytisine in the adhesive layer, and high skin permeability of cytisine. From the description in Table 1, it is apparent that the patches comprising an acrylic adhesive as the adhesive have good releasing property of cytisine from the adhesive layer and high permeability of cytisine; that the patches comprising a rubber adhesive as the adhesive have high stability of cytisine in the adhesive layer; and that the hydroxyl group-containing acrylic adhesive significantly enhances the releasing property of cytisine from the adhesive layer in the patch. In Table 1, “skin permeability (C.A. (μg/cm²))” is a cumulative permeation amount of cytisine until 24 hours.

### Example 6

A patch was obtained in the same manner as in Example 4 except that polyacrylic acid (PAA) as an organic acid was added to the adhesive composition solution in Example 4.

### Example 7

A patch was obtained in the same manner as in Example 6 except that PAA was replaced by alginic acid as an organic acid.

### Example 8

A patch was obtained in the same manner as in Example 6 except that PAA was replaced by aspartic acid as an organic acid.

### Example 9

A patch was obtained in the same manner as in Example 6 except that PAA was replaced by glutamic acid as an organic acid.

### Example 10

A patch was obtained in the same manner as in Example 6 except that PAA was replaced by sodium laurate as a salt of an organic acid.

The adhesive layers of the patches in Examples 6 to 10 each comprise 3% by weight of cytisine, 94% by weight of the adhesive (SIS5002: 27.7% by weight, alicyclic hydrocarbon resin: 49.8% by weight, liquid paraffin: 16.5% by weight), and 3% by weight of the organic acid or the salt thereof relative to the total weight of the adhesive composition.

The acid value, the releasing property, and the stability of the patches in Examples 6 to 10 are shown in Table 2.

TABLE 2

| | | | Releasing | Stability |
|---|---|---|---|---|
| | | | property (24 hr | (to |
| | Organic acid or | Acid value | release rate into | initial |
| | salt thereof | (mgKOH/g) | water (%)) | content %) |
| Example 6 | Polyacrylic | 1 | 42.4 | 97.8 |
| | acid | | | |
| Example 7 | Alginic acid | 1 | 24.2 | 98.4 |
| Example 8 | Aspartic acid | 1 | 18.7 | 99.2 |
| Example 9 | Glutamic acid | 1 | 21.7 | 99.8 |
| Example 10 | Sodium laurate | 0 | 17.1 | 101.4 |

From Table 2, it is apparent that if an organic acid or a salt thereof is comprised in an adhesive composition comprising a rubber adhesive, the releasing property of cytisine from the adhesive layer are enhanced.

### Example 11

A patch was obtained in the same manner as in Example 1 except that an absorption enhancer isopropyl palmitate (IPP) was added to the adhesive composition solution in Example 1.

### Example 12

A patch was obtained in the same manner as in Example 11 except that IPP was replaced by diethyl sebacate as an absorption enhancer.

### Example 13

A patch was obtained in the same manner as in Example 11 except that IPP was replaced by octyldodecanol as an absorption enhancer.

### Example 14

A patch was obtained in the same manner as in Example 11 except that IPP was replaced by dipropylene glycol as an absorption enhancer.

### Example 15

A patch was obtained in the same manner as in Example 11 except that IPP was replaced by triacetin as an absorption enhancer.

### Example 16

A patch was obtained in the same manner as in Example 11 except that IPP was replaced by propylene glycol monolaurate as an absorption enhancer.

### Example 17

A patch was obtained in the same manner as in Example 11 except that IPP was replaced by dimethyl sulfoxide as an absorption enhancer.

The adhesive layers of the patches in Examples 11 to 17 each comprise 3% by weight of cytisine, 92% by weight of the adhesive, and 5% by weight of the absorption enhancer relative to the total weight of the adhesive composition.

The acid value and the skin permeability of the patches in Examples 11 to 17 are shown in Table 3. The value of “To control” in the right column in Table 3 represents the value obtained by dividing each C.A. value (μg/cm²) in Examples 11 to 17 by the C.A. value (78.73 μg/cm²) in Example 1, and indicates how many times the skin permeability is enhanced compared to that of the patch in Example 1. In Table 3, “C.A. (μg/cm²)” is a cumulative permeation amount of cytisine until 24 hours.

TABLE 3

| | | Acid value | C.A. | |
|---|---|---|---|---|
| | Absorption enhancer | (mgKOH/g) | (μg/cm²) | To control |
| Example 11 | Isopropyl palmitate | 1 | 226.29 | 2.9 |
| Example 12 | Diethyl sebacate | 1 | 163.90 | 2.1 |
| Example 13 | Octyldodecanol | 1 | 178.43 | 2.3 |
| Example 14 | Dipropylene glycol | 1 | 108.37 | 1.4 |
| Example 15 | Triacetin | 1 | 139.52 | 1.8 |
| Example 16 | Propylene glycol | 2 | 213.83 | 2.7 |
| | monolaurate | | | |
| Example 17 | Dimethyl sulfoxide | 2 | 152.98 | 1.9 |

From Table 3, it is apparent that if an absorption enhancer is comprised in the adhesive composition comprising a hydroxyl group-containing acrylic adhesive, the skin permeability of cytisine is enhanced.

1. A patch comprising a support, and an adhesive layer disposed on at least one surface of the support and formed of an adhesive composition comprising a drug and an adhesive,
wherein the drug is cytisine or a salt thereof, and the adhesive composition has an acid value of 10 or less.

2. The patch according to claim 1, wherein the adhesive is an acrylic adhesive.

3. The patch according to claim 1, wherein the adhesive is a hydroxyl group-containing acrylic adhesive.

4. The patch according to claim 1, wherein the adhesive is a rubber adhesive.

5. The patch according to claim 1, wherein the adhesive composition further comprises an organic acid or a salt thereof.

6. The patch according to claim 1, wherein the adhesive composition further comprises an absorption enhancer.

7. The patch according to claim 2, wherein the adhesive composition further comprises an organic acid or a salt thereof.

8. The patch according to claim 3, wherein the adhesive composition further comprises an organic acid or a salt thereof.

9. The patch according to claim 4, wherein the adhesive composition further comprises an organic acid or a salt thereof.

10. The patch according to claim 2, wherein the adhesive composition further comprises an absorption enhancer.

11. The patch according to claim 3, wherein the adhesive composition further comprises an absorption enhancer.

12. The patch according to claim 4, wherein the adhesive composition further comprises an absorption enhancer.

13. The patch according to claim 5, wherein the adhesive composition further comprises an absorption enhancer.

14. The patch according to claim 7, wherein the adhesive composition further comprises an absorption enhancer.

15. The patch according to claim 8, wherein the adhesive composition further comprises an absorption enhancer.

16. The patch according to claim 9, wherein the adhesive composition further comprises an absorption enhancer.
